(19) United States
(12) Patent Application Publication
(10) Pub. No.: US 2009/0137922 A1
(43) Pub. Date: May 28, 2009

(54) METHOD FOR ANTI-STRESS TRAINING

(75) Inventor: Staffan Soderlund, Kista (SE)

Correspondence Address: MORRISON & FOERSTER LLP, 425 MARKET STREET, SAN FRANCISCO, CA, 94105-2482, US

(73) Assignee: INTERACTIVE PRODUCTLINE AB, Kista (SE)

(21) Appl. No.: 12/095,115

(22) PCT Filed: Nov. 27, 2006
(86) PCT No.: PCT/US06/45448

§ 371 Date: Aug. 22, 2008

Related U.S. Application Data
(60) Provisional application No. 60/597,392, filed on Nov. 29, 2005.

(51) Int. Cl. A61B 5/0476; A63B 43/00

(52) U.S. Cl. 600/544; 473/569

(57) ABSTRACT

The method is for reducing a stress level of a user. The user is provided with a sensor (23) of a headband (20) for measuring brain wave frequencies of the user (28). The user (28) moves a unit (24), such as a ball, on a surface (32) or in a computer screen (104) along a path (16) towards a goal point (18) when a brain wave frequency (26) of the user (28) is lowered to be within a preferred frequency range (15) and towards a starting point (14) when the brain wave frequency (26) is outside the frequency range (15). The computer may be connected to the Internet so the user may interact with other users via the Internet.

### TECHNICAL FIELD

The present invention relates to a method for anti-stress training. More particularly, the method is a training concept to help the user better handle stress and enhance the ability to relax, focus and concentrate.

### BACKGROUND OF THE INVENTION

In today's society people are becoming more and more stressed due to increased pace in life. Some of the stress is related to increased use of the brain at relatively high wave frequencies such as when a person is agitated. The injuries and reduced quality of life related to stress could be reduced if people could improve their ability to control the brain wave frequencies so that the brain operates more at lower brain wave frequencies. There is a need for an effective and stimulating way of learning to control the brain wave frequencies to, among other things, lower the stress level of the brain and thus the body of the user. There is also a need for a method to enhance the user's ability to concentrate and focus.

### SUMMARY OF THE INVENTION

The method of the present invention provides a solution to the above-outlined problems. More particularly, the method of the present invention is for training the user to reduce a stress level of the user. The user is provided with sensors on a headband for measuring brain wave frequencies of the user. The sensors are in communication with a processor of an electronic device on which a ball is placed. The user moves the ball on a surface along a path towards a goal point when a brain wave frequency of the user is within a preferred frequency range, such as 4-12 Hz, and towards a starting point when the brain wave frequency is outside this preferred frequency range.

The method of the present invention is not limited to moving physical objects. The sensors can also communicate with a computer connected to a screen in order to control and virtually move objects on the screen. The computer may be connected to the Internet and the user may interact via the Internet with other users.

### BRIEF DESCRIPTION OF THE DRAWING

FIG. 1 is a schematic view of a training product of the present invention; and

FIG. 2 is a schematic view of an alternative embodiment of the present invention.

### DETAILED DESCRIPTION

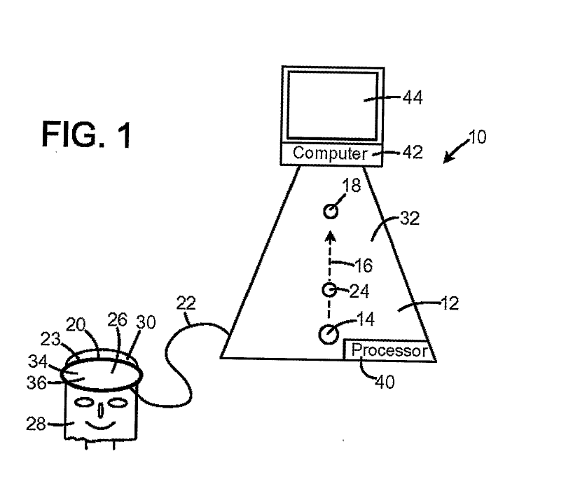

With reference to FIG. 1, the method and device 10 of the present invention includes an electronic device 12 that has a starting point 14, a path 16 and a goal or end point 18. A headband 20 is electrically connected to the device 10 via a wire 22. The headband 20 may be electrically connected to or in communication with the device 12 by wireless technology, if desired. The headband 20 may have biosensor units 23 mounted thereon for measuring brainwaves of a brain of a user 28.

A magnetic ball 24 or any other suitable item may be placed on the device 10 such as on the starting point 14. The ball 24 may be moved between the starting point 14 and the goal point 18 depending upon brain-wave frequencies 26 of the user 28 as measured by the sensors units 23 in the headband 20 when the headband is put over a head 30 of the user 28.

The ball 24 may roll on the surface 34. The ball 24 may also float a distance above an upper surface area 32 in a z-direction that gives an illusion of zero gravity.

Preferably, the movement of the ball 24 in the z-direction is not a variable. However, in a three dimensional variation of the device 10, the z-direction is a variable also.

In operation, the user 28 places the headband 20 over the user's head 30. The device 12 is placed on an even surface such as a table. The user 28 may sit beside or behind the device so that the user can see ball 24 placed on the device 10. With the headband 20 placed on the head 30 of the user 28, the brain wave frequencies 26 of the user 28 may move the ball 24 towards the goal point 18 or towards the start point 14. For example, theta wave frequencies 34 and alpha wave frequencies 36 of the user 28 are measured by the sensors 23 and sent to a processor 40 via the wire 22 for processing. The theta wave frequencies 34 may range from 4-8 Hz while the alpha wave frequencies 36 may range from 8-12 Hz. In general, the lower the frequencies the calmer the user is. The sensors 23 may be connected to a biosensor system that registers the electrical activity of the brain of the user 28. Electro-Encephalography (EEG) may be used to measure the brain wave frequencies.

The processor 40 may be programmed so that the ball 24 moves from the starting point 14 towards the end point 18 when the user's alpha wave frequencies in within a preferred frequency range 15 such as between 8-12 Hz. It is to be understood that any suitable preferable frequency range may be used such as the user's range of alpha wave frequencies. This range may also include the lower theta wave frequencies or any other suitable frequencies such as frequencies greater than 12 Hz.

In other words, the calmer the user is so that the user's brain frequency is within the range 15 then the ball 24 moves on the path 16 towards the goal point 18. It is also possible to control the speed of the ball 24 so that the ball 24, for example, has a first high velocity when the brain wave frequency is closer to 8 Hz or lower and a second lower velocity when the brain wave frequency is approaching 12 Hz or higher so that the velocity of the ball 24 increases with reduced brain wave frequency. When the user 28 loses concentration or get agitated the brain wave frequency 36 increases and the ball 24 starts to roll back to the starting point 14 until the user has managed to lower the brain wave frequencies back to the range 15 again.

It is possible to connect the unit 40 to an external computer 42 including a monitor 44 that shows the brain wave frequency of the user.

By learning to control the brain wave frequency of the brain by biofeedback, the user may better be able to put him/her in a more relaxed condition and enhance the ability to concentrate and focus on tasks at hand.

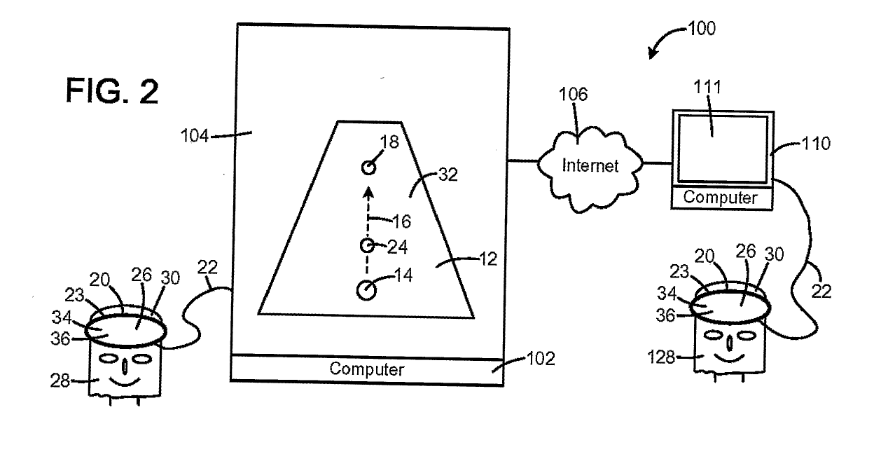

FIG. 2 shows an alternative system 100 that is virtually identical to the device 10 except that the user is connected to a computer 102 so that the features and movements are shown on a computer screen 104 instead. The computer 102 may also be connected to the Internet 106 so that a second user 128, connected to a computer 110 with a monitor 111, may interact with the user 28. The view on the screen of the second user is virtually identical or identical to the view of the screen of the first user.

While the present invention has been described in accordance with preferred compositions and embodiments, it is to be understood that certain substitutions and alterations may be made thereto without departing from the spirit and scope of the following claims.

1. A method of training to reduce a stress level, comprising: providing a user (28) with a sensor (23) for measuring brain wave frequencies of the user (28);
the user (28) moving a unit (24) on a surface (32) towards a goal point (18) when brain wave frequencies (26) of the user (28) are within a preferred frequency range (15) and towards a starting point (14) when the brain wave frequencies (26) are outside the preferred frequency range (15).

2. The method according to claim 1 wherein the method further comprises mounting the sensors (23) on a headband (20) that is placed over a skull (30) of the user (28).

3. The method according to claim 1 wherein the method further comprises the user (28) moving a ball.

4. The method according to claim 1 wherein the method further comprises measuring alpha wave frequencies (36) of a brain of the user (28).

5. The method according to claim 1 wherein the method further comprises the user (28) increasing a velocity of the unit (24) by lowering the brain wave frequencies (26) of the user (28) to the preferred frequency range (15).

6. The method according to claim 1 wherein the method further comprises displaying brain wave frequencies (26) on a display (44).

7. The method according to claim 3 wherein the method further comprises moving the ball along a path (16) away from the user (28) when the brain wave frequencies (26) are lowered to be within the preferred frequency range (15).

8. The method according to claim 1 wherein the method further comprises sending information from the sensor (23) to a processor (40 by wireless technology.

9. The method according to claim 1 wherein the method further comprises setting the preferred frequency range (15) to 4-12 Hz.

10. A method of training to reduce a stress level, comprising:
providing a user (28) with a sensor (23) for measuring brain wave frequencies of the user (28), the sensor (23) being connected to a computer (102) that has a screen (104);
the user (28) virtually moving a unit (24) on the screen (27) towards a goal point (18) on the screen when brain wave frequencies (26) of the user (28) are within a preferred frequency range (15) and towards a starting point (14) on the screen when the brain wave frequencies (26) are outside the preferred frequency range (15).

11. The method according to claim 10 wherein the method further comprises the user (28) moving objects on the screen.

12. The method according to claim 10 wherein the method further comprises connecting the computer to the Internet (106) and the user interacting with a user (128) via the Internet.
